(19) United States
(12) Patent Application Publication
(10) Pub. No.: US 2009/0298915 A1
(43) Pub. Date: Dec. 3, 2009

(54) TOPICAL DRUG DELIVERY

(76) Inventors: Eugene Pius Joseph Healy, Southampton (GB); Peter Simon Friedmann, Awbridge (GB); Mark Bradley, Edinburgh (GB)

Correspondence Address: MORRISON & FOERSTER LLP, 12531 HIGH BLUFF DRIVE, SUITE 100, SAN DIEGO, CA, 92130-2040, US

(21) Appl. No.: 12/295,603

(22) PCT Filed: Mar. 30, 2007
(86) PCT No.: PCT/GB2007/001193

§ 371 Date: Mar. 27, 2009

(30) Foreign Application Priority Data
Mar. 31, 2006 (GB) 0606415.8

(51) Int. Cl. A61K 31/7088; A61K 47/42; C07K 5/08; A01N 1/02

(52) U.S. Cl. 514/44 A; 514/773; 530/331; 435/1.1

(57) ABSTRACT

Poly-pseudo-lysine conjugates have been shown to be able to penetrate into human skin and are proposed for both therapeutic and cosmetic treatments by topical application, e.g. change of skin pigmentation.

The present invention relates to topical drug delivery. More particularly, it relates to the finding that a known type of cell penetrable poly-peptoid, poly-pseudo-lysine, can be used as a carrier to enable cutaneous drug penetration. Such drug delivery is of interest for various therapeutic treatments, especially, for example, treatment of skin disorders, but may also be applied for purely cosmetic purpose to alter skin characteristics such as degree of tanning.

### BACKGROUND OF THE INVENTION

There are a number of benefits to treating skin disorders with topically applied agents rather than by systemic (oral/intravenous/subcutaneous/intramuscular) agent administration. For example, (i) systemic adverse effects of the drug may be avoided or reduced, (ii) metabolism and inactivation of the drug by the liver may be circumvented, and (iii) higher local concentrations of the drug may be attained than would be safely possible by systemic administration. However, the skin offers a formidable barrier to the delivery of a wide range of therapeutic agents owing to the outer layers of the stratified epithelium (the stratum corneum). With the exception of topical corticosteroids, many drugs do not adequately penetrate into skin to enable their use topically. This is the case for drugs commonly used to treat human and animal skin diseases. Significant benefits could also come from administering systemic therapeutic agents through the skin for treatment of diseases of other organ systems.

Several polypeptides have previously been reported to penetrate into cells in culture, e.g. the Tat protein transduction domain, penetratin/antennopedia and polylysine, but only a few such cell penetrable polypeptides are known to be capable of penetrating into skin (reviewed in Dietz & Bahr, Mol. Cell. Neurosci. (2004) 27, 85-131). To date only one such polypeptide, a heptamer of arginine, has been reported to penetrate into human skin and was further shown to be capable of carrying into skin cyclosporin A and other drugs (Rothbard et al. Nature Med. (2000) 6, 1253-1257). A few others, including polylysine and the Tat transduction sequence, have been shown to be capable of carrying molecules into pig and/or mouse skin (Park et al., Mol. Cells (2002) 13, 202-208; Lopos et al. Pharm. Res. (2005) 22, 750-757). However, such cell penetrable polypeptides have disadvantages for clinical use as drug carriers. There is desire for alternative carriers for topical drug delivery which are both convenient to synthesize and less susceptible to in vivo modification.

It has previously been reported that poly-peptoid oligomers with lysine-type side chains (3 to 7 mers) are capable of entering cells in culture conjugated to fluorescein as a fluorescent label (Peretto et al., Chem. Commun. (2003) 2312-2313). However, such studies do not enable extrapolation that the same type of poly-peptoid structure (poly-pseudo-lysine, PPL) can act as a carrier for enabling cutaneous penetration of any drug. Using two different forms of attachment to PPL, it has now been established, however, that the tridecapeptide alpha-melanocyte stimulating hormone (αMSH) conjugated to a 7 mer PPL will penetrate into both cells in culture and mouse, pig and human skin ex vivo with retention of ability to increase pigmentation through binding to the melancortin 1 receptor (MC1R). In addition, it has been shown that an antisense peptide nucleic acid (PNA) conjugated to the same PPL can penetrate into cells and skin and inhibit tyrosinase activity and melanin pigment production. The same is extrapolated to be the case for a 19 mer antisense oligonucleotide conjugated to PPL on the basis of cellular studies reported herein. Such findings open the way to a new form of therapeutic treatment relying on PPL as a carrier for delivering therapeutic agents into skin. PPL has advantage over poly-arginine for this purpose in view of its ease of synthesis by solid phase chemistry combined with greater resistance to in vivo degradation.

### SUMMARY OF THE INVENTION

PPL as previously synthesised is formed of units of formula Ia

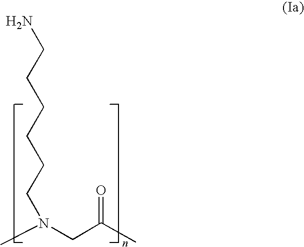

However, it is anticipated that various variants of this basic unit may be substituted with retention of cell penetration ability as indicated by the more generalised structure I below.

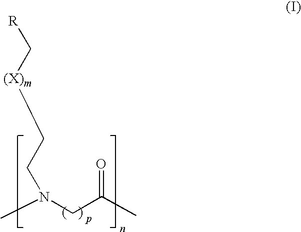

wherein:
n=3 or more, more preferably 6 or more, e.g. 6 to 9, most preferably 7
p=1 or more, preferably 1, 2 or 4;
X=either CH₂, in which case m=1 or more, e.g. 1 to 5, preferably 3 such that the R group is attached by a hexane side chain; or
   CH₂CH═CH and m=1; or
   CH═CH and m=1; or
   CH₂CC and m=1; or
   CC and m=1; and
R=a group selected from primary amine (NH₂), guanidinium (NHC(═NH)—NH₂), amidine (C(═NH)NH₂),
   a secondary amine (NHA, where A=alkyl, e.g. Me, Et, nPr, Bn, preferably lower alkyl, e.g alkyl 1-4, most preferably Me or Et),
   a tertiary amine (NA¹A², where A¹ and A² may be the same or different and are alkyl, e.g. Me, Et, nPr, Bn, preferably Me or Et), or
   a quaternary amine (N⁺A¹A² A³ wherein each of A¹, A² and A³ may be the same or different and are alkyl, e.g. Me, Et, nPr, Bn, preferably Me or Et).

In one aspect of the invention, there is thus provided use of a drug conjugate in which the drug is conjugated to a poly-peptoid having at least 3 units of formula I as given above in the manufacture of a therapeutic composition for use in delivering said drug into skin by topical application. The term “therapeutic composition” will be understood to refer to a pharmaceutical composition in which the conjugate is combined with a pharmaceutically acceptable carrier to provide, for example, a cream, ointment, lotion or gel composition, but will also be understood to refer to a patch or plaster incorporating the conjugate in releasable form which can be applied to skin.

It is extrapolated that the poly-peptoid carrier may preferably for example have units selected from the following:
(a) units of formula I wherein n is as above, R=primary amine, guanidinium or amindine, preferably primary amine, attached by a hexane chain and p=1, 2 or 4; or
(b) units of formula I wherein n is as above, R=a secondary amine attached by a hexane chain and p=1, 2 or 4; or
(c) units of formula I wherein n is as above, R=primary amine, guanidinium or amindine, preferably primary amine, attached by a hexane chain and p=1.

The poly-peptoid carrier may, however, most preferably be a poly-pseudo-lysine of the form previously described having units of formula Ia. In a particularly preferred embodiment of the invention, there is thus provided use of a drug conjugate in which the drug is conjugated to poly-pseudo-lysine having at least 3 units of formula Ia

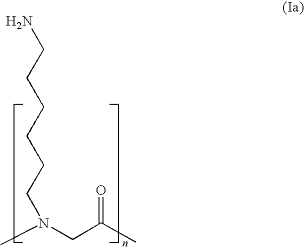

in the manufacture of a therapeutic composition for use in delivering said drug into skin by topical application. Preferably, n is more than 3, more preferably 6 or more, most preferably 7. For the skin penetration studies presented herein, it was found convenient to use PPL oligomers of 7 units, which as fluorescently labelled conjugates showed improved uptake into cells in culture compared with a 3 mer or 5 mer.

In another aspect, there is provided a method of delivering an agent into skin ex vivo comprising:
(i) providing a conjugate in which the agent is conjugated to a poly-peptoid as above, preferably a poly-pseudo-lysine oligomer as above, said conjugate additionally carrying a label detectable in skin, e.g. a fluorescent label;
(ii) applying said conjugate to the cutaneous surface of a skin sample ex vivo, and
(iii) determining whether the labelled conjugate penetrates into the skin sample.

Such a method may be used, for example, to test agents for ability to change skin characteristics.

As indicated above, the invention also provides new treatments which may be applied for cosmetic rather than therapeutic purpose to change skin characteristics. Thus, in a still further aspect, there is provided cosmetic use of a conjugate in which an agent suitable for changing a skin characteristic is conjugated to a poly-peptoid as above, preferably a poly-pseudo-lysine as above, and said conjugate is applied to skin whereby said conjugate penetrates into the skin and said agent is effective to change said skin characteristic, e.g. skin pigmentation.

For this purpose, the agent coupled to the poly-peptoid may preferably be αMSH or an active derivative thereof that targets the MCIR. Topically applied αMSH fails to penetrate human skin. The finding that αMSH can be delivered into skin using a PPL carrier as an active agent and enter cells thus opens up important new uses of the polypeptide and active derivatives thereof both in the cosmetic industry and clinically. Alternatively, the agent conjugated to the poly-peptoid may be an agent which down regulates tyrosinase expression and thereby reduces skin pigmentation, for example, a peptoid nucleic acid (PNA) antisense molecule or antisense oligonucleotide for targeting tyrosinase mRNA.

Cosmetic preparations and pharmaceutical compositions adapted for topical application including a poly-peptoid conjugate as described above constitute yet further aspects of the invention.

Previously proposed poly-pseudo-lysine conjugates have a terminal NH₂ group with linkage at the other terminus to a second component via a six carbon aminohexanoic spacer unit as shown by general formula II below, but the invention is not limited to such a spacer; any spacer may be employed which retains the desired skin penetration.

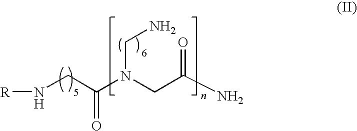

Such a conjugate in which a 7 mer poly-pseudo-lysine oligomer is linked to αMSH (designated αMSH-PPL[98]) is shown in FIG. 1 and more schematically by formula III below.

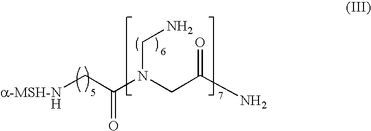

While this conjugate labelled with a fluorescent fluorescein group (see FIG. 2) has been shown to penetrate into human skin ex vivo, it has been found that by using a different attachment of αMSH to a PPL oligomer of the same length (see FIG. 3) better skin penetration can be observed. A PPL conjugate having the structure shown in FIG. 3 (designated αMSH-PPL [99]) or a variant thereof wherein αMSH is substituted by an active derivative thereof or another agent in accordance with general formula IV below, wherein n=6 or more, most preferably 7, constitutes a still further aspect of the invention:

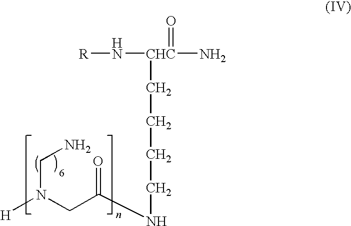

Formula V shows more schematically the preferred conjugate of general formula IV wherein R=αMSH and n=7.

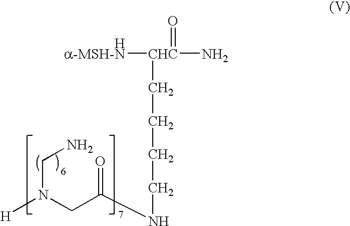

The invention also extends to use of variants of PPL conjugates as described above in which the linkage of the active agent to the PPL carrier is substituted by an alternative linkage, for example, a linkage cleavable in skin such as an esterase cleavable linkage, a disulphide bond or photolytic linkage. The same linkages may be used with alternative poly-peptoid carriers.

Formula VI below shows a preferred PPL conjugate for transport of a PNA into skin.

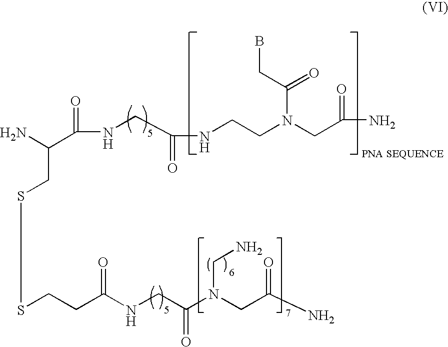

where B is a base of the PNA sequence. The PNA may, for example, be a PNA capable of targeting tyrosinase expression in skin, e.g. a 15 mer as noted in Example 2 or an equivalent PNA which complements the human tyrosinase coding sequence.

Formula VII below additionally shows a PPL conjugate which is anticipated to be useful in transporting DNAs, e.g. antisense oligonucleotides, into skin wherein the oligonucleotide is linked to the PPL via a disulphide bond.

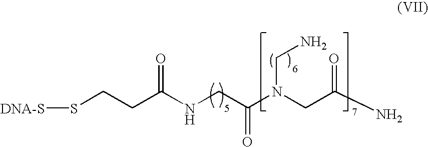

### BRIEF DESCRIPTION OF THE FIGURES

The invention will be further described below with reference to the following figures.

FIG. 1: Structure of αMSH-PPL[98]

FIG. 2: Structure of fluorescein-labelled αMSH-PPL[98]

FIG. 3: Structure of αMSH-PPL[99]

FIG. 4: Structure of fluorescein-labelled αMSH-PPL[99]

FIG. 5: Alternative linkers for conjugation of therapeutic agents to PPL oligomers; (a) disulphide linker; (b) sulphide linker; (c) and (d) photolytic linkers; (e) and (f) esterase cleavable linkers.

FIG. 6: Scheme for synthesis of αMSH-PPL[99]

FIG. 7: Scheme for synthesis of TyrPNA in which the PNA sequence is the 15 mer PNA of SEQ ID no. 1 which targets murine tyrosinase.

FIG. 8: Scheme for synthesis of fluorescently-labelled PNA of SEQ. ID no. 1 (TyrPNA-Fluo)

FIG. 9: Scheme for synthesis of a TyrPNA-PPL conjugate in which the PNA sequence is the PNA of SEQ. ID. no. 1

FIG. 10: Scheme for synthesis of a fluorescently-labelled TyrPNA-PPL conjugate in which the same PNA sequence is present.

In any of the above FIGS. 7 to 10, the PNA sequence may be substituted by an alternative PNA sequence of interest for topical delivery in accordance with the invention

FIGS. 11 and 12: Additionally provide schemes for synthesis of oligonucleotide-PPL conjugates, e.g. the DNA may be an anti-sense oligonucleotide as illustrated by Example 2.

### DETAILED DESCRIPTION OF THE INVENTION

Therapeutic Treatment

The use of poly-peptoid oligomers as described above, especially PPL oligomers, as carriers for transporting therapeutic agents into skin is evidently of particular interest in relation to treatment of skin disorders, e.g. inflammatory skin disorders such as psoriasis and contact dermatitis, eczema, skin cancer and pigmentation disorders. However, since a poly-peptoid conjugate (or drug released therefrom in vivo) may penetrate human skin down to the dermis and through the skin, it is also envisaged that such conjugates may have wider therapeutic application.

As indicated above, the poly-peptoid carrier of the conjugate will generally be a short oligomer having 3 to about 7 units of general formula I, preferably general formula Ia, as shown above, preferably 6 or 7 units, conveniently and preferably 7 units. In the studies of Peretto et al. reported in Cell. Commun. 2003, 2312-2313, the ability of fluorescein-labelled PPL oligomers of n=3, 5 and 7 to enter cells in culture was found to increase with peptoid oligomer length.

The therapeutic agent linked to the poly-peptoid carrier may, for example, be a polypeptide, e.g. a hormone such as αMSH or insulin, a peptide such as a peptide vaccine, an enzyme, e.g. an anti-oxidant enzyme such as superoxide dismutase, a molecule which acts extracellularly or intracellularly to affect cellular function or augment or inhibit cell signalling or augment or inhibit gene transcription/translation, a nucleic acid sequence or a peptide nucleic acid (PNA). It is envisaged that a poly-peptoid carrier as above may also be linked to a therapeutic polynucleotide such as a DNA vaccine, an anti-sense polynucleotide, a siRNA or polynucleotide for gene therapy. It is additionally envisaged that a poly-peptoid carrier as above may be used to deliver an antibody fragment into the skin or even perhaps a whole antibody. Other drugs which may desirably be administered in accordance with the invention include methotrexate (MTX) and other drugs useful in the treatment of skin disorders such as azathioprine, hydroxycarbamide and thalidomide.

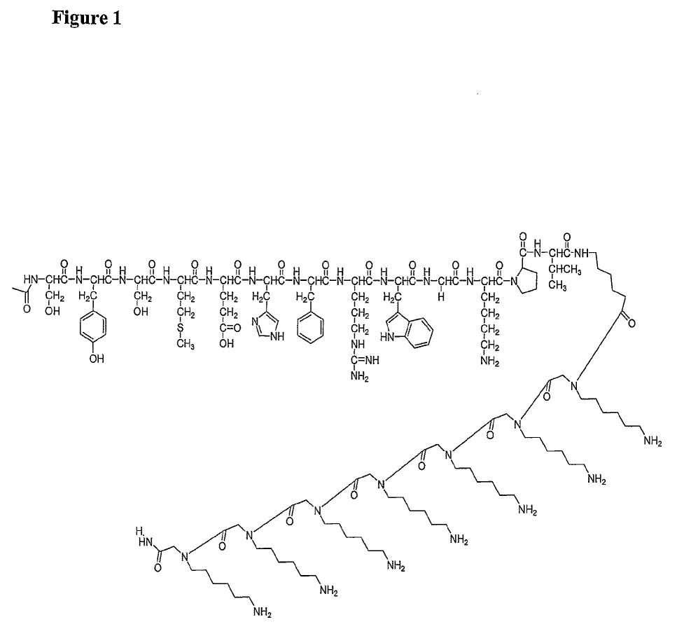

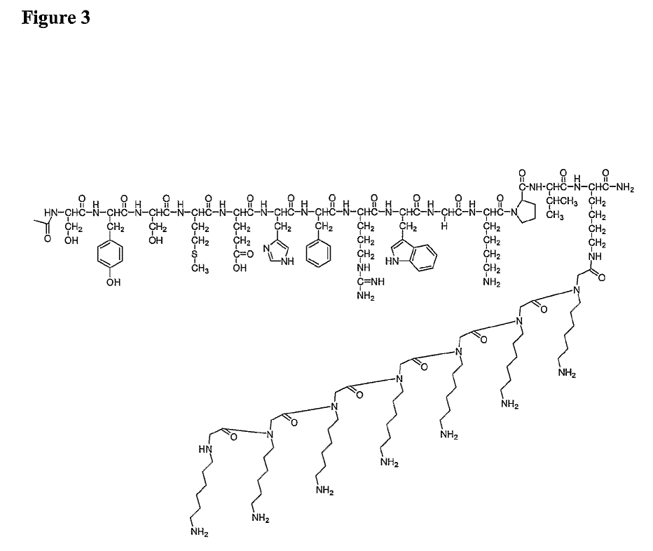

As already noted above, the invention stemmed from investigation of skin penetration by two different types of PPL conjugate as shown in FIGS. 1 and 3 in which a PPL oligomer of 7 units is conjugated to αMSH. αMSH has the potential to switch on pigmentation in skin and also to act as an immunosuppressive and anti-inflammatory agent. Significantly, αMSH and derivatives thereof are known to have both important immunomodulatory and anti-inflammatory properties. Studies with mice showed that such compounds will suppress cutaneous hypersensitivity reactions to allergens/contact dermatitis. More recently, it has also been demonstrated that αMSH can potently suppress antigen-induced lymphocyte proliferation in vitro; in assays with antigen-induced stimulation indices of up to 100-fold, αMSH caused a reduction in lymphocyte proliferation of up to 80%. However, the need for systemic administration of αMSH and active derivatives thereof has meant that up to now use of such agents in relation to treatment of skin disorders has not been favoured. Use of a PPL oligomer (or variant poly-peptoid as above) conjugated to αMSH or an active derivative thereof for provision of a therapeutic composition for topical application is thus envisaged as an important addition to the armoury of treatments available for tackling a variety of skin disorders.

Importantly, αMSH coupled to PPL as described above has been shown to retain the ability to bind to MC1R and stimulate pigmentation of melanoma cells. Thus, poly-peptoid conjugates as above in which the poly-peptoid is linked to αMSH, or an active derivative thereof, e.g. Nle4, D-Phe7 αMSH, melanotan II (MTII), SHU9119, are envisaged as having use both therapeutically and for entirely cosmetic purpose. Cosmetic use may overlap with therapeutic use but may be entirely independent of any therapeutic use and applied to healthy individuals as will be discussed in more detail below.

Conjugation of MTX to poly-L-lysine was previously shown to increase transport into cells in culture (Ryser et al., Proc. Natl. Acad. Sci. USA (1978) 75, 3867-3870). It is therefore now also to be expected that MTX linked to a PPL oligomer (or poly-peptoid variant thereof as above) will enable its administration topically, e.g. for treatment of psoriasis, thereby avoiding or at least reducing the side effects associated with systemic administration. Systemic administration of MTX has been widely used for patients with generalised psoriasis which is recalcitrant to conventional topical therapies and ultraviolet radiation. The evidence that MTX is effective in this scenario is overwhelming. However, the risk of haematopoetic suppression and liver fibrosis means that long term monitoring is essential. Indeed, the systemic use of MTX can lead to fatalities, with elderly patients and those with renal insufficiency more prone to MTX-induced adverse effects. Furthermore, MTX is contra-indicated during pregnancy and breast-feeding. Nevertheless. MTX is also employed, although less frequently, for a variety of other skin disorders, including cutaneous sarcoid, morphoea and eczemas which are refractory to other therapies. Hence, use of MTX-poly-peptoid conjugates in accordance with the invention offers the prospect of less problematic MTX therapy and greater benefit of MTX in treating dermatological conditions.

Amongst other preferred envisaged uses of poly-peptoid carrier conjugates in accordance with the invention, is use of a poly-peptoid carrier to transport DNA repair molecules into skin to repair ultraviolet light-induced damage in patients with xeroderma pigmentosum (and into skin of normal individuals who have had significant recent sun-exposure). The chosen DNA repair molecule may for example be the bacterial DNA repair enzyme T4 endonuclease V. It has previously been reported that T4 endonuclease V applied in liposomes to skin of patients with xeroderma pigmentosum lowered the rate of new skin cancers in such patients (Yarosh et al. Lancet (2001) 357, 926-929).

Poly-peptoid carriers in accordance with the invention conjugated to PNAs or antisense oligonucleotides may have many therapeutic uses through skin penetration, particularly in reducing or preventing inflammation, e.g. blocking the effects of pro-inflammatory cytokines such as TNFalpha. A PNA-PPL conjugate capable of targeting TNFalpha expression is especially of interest as an anti-psoriasis agent. PNA-PPL conjugates, like αMSH-PPL conjugates, are equally of interest in relation to altering skin pigmentation as discussed further below. Of particular interest for this purpose are PNA-PPL conjugates which can reduce tyrosinase expression in skin and thereby reduce pigmentation (see Example 2); this is likely to have therapeutic and cosmetic applications.

Reference has previously been made above to possible use of poly-peptoid oligomers in accordance with the invention to carry enzymes into skin. This is of particular interest for example for provision of anti-oxidant activity. Superoxide dismutase (SOD) is among the key cellular enzymes by which cells detoxify free radicals and protect themselves from oxidative damage and hence is considered of particular interest for delivery into skin to protect against reactive oxygen species damage. Polylysine was previously shown to be able to transduce SOD into mouse skin (Park et al., Mol. Cells. 13, 202-208). Poly-peptoid oligomers in accordance with the invention, especially PPL oligomers, are now proposed as a more advantageous carrier for the same purpose in both animal and human skin. It is envisaged that such SOD fusion constructs may be employed for therapeutic purpose but also in cosmetics to combat oxidative damage in skin with aging.

The above-described uses of poly-peptoid oligomers as carriers for drug transportation into skin are provided by way of illustration. Many others will be apparent to clinicians seeking to avoid problems associated with systemic administration of drugs for a wide variety of conditions.

### Conjugates and Pharmaceutical Compositions

Pharmaceutical compositions adapted for topical application and suitable for therapeutic treatment as discussed above are now provided. Such compositions will comprise a desired poly-peptoid conjugate together with a carrier to facilitate topical application. Such a composition may be, for example, in the form of a cream or gel. As previously indicated, therapeutic compositions of the invention also extend to patches and plasters for application to skin and incorporating the desired drug conjugate in a form such that it will be released into the skin.

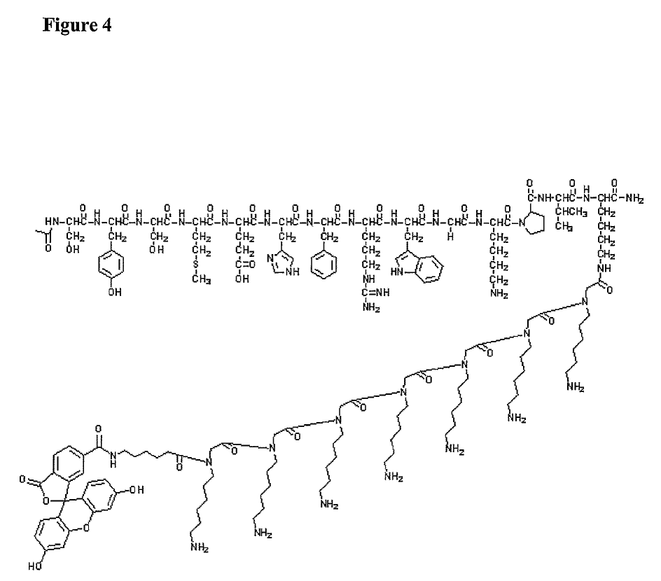

The poly-peptoid conjugate may preferably be a PPL conjugate of general formula II as above, e.g. αMSH-PPL[98] a shown in FIG. 1 or a variant thereof wherein the αMSH is substituted by an active derivate thereof or an alternative agent. Alternatively, the PPL conjugate will be a PPL conjugate of general formula IV as above, e.g. preferably αMSH-PPL [99] as shown in FIG. 3 or a variant thereof wherein αMSH is substituted by an active derivative thereof or another agent. As indicated above, such a preferred conjugate which is favoured for skin penetration constitutes a still further aspect of the invention. For use in screening tests as described further below, such a conjugate will be labelled with a label detectable in skin, e.g. a fluorescent label. This may be a fluorescein group attached to the end of the PPL oligomer distant from the conjugated agent as exemplified by αMSH-PPL[99]-fluo shown in FIG. 4.

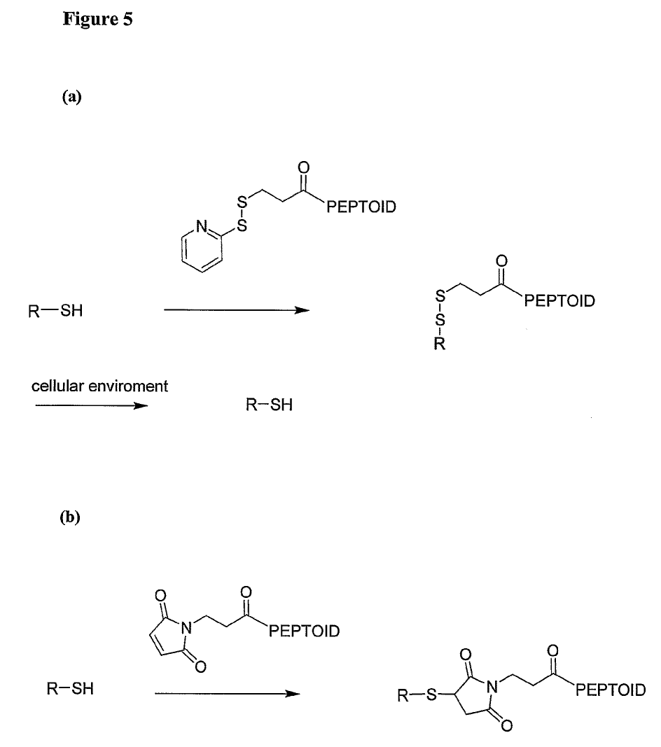

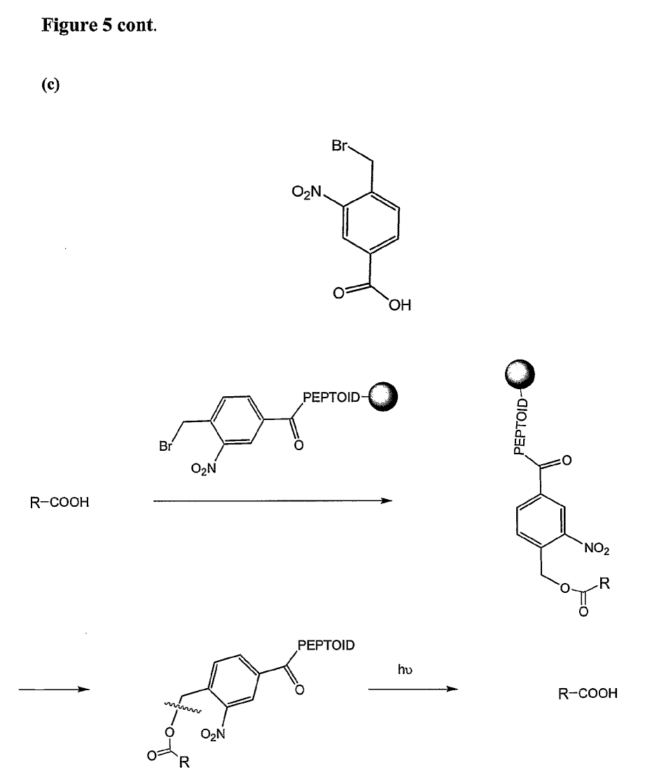

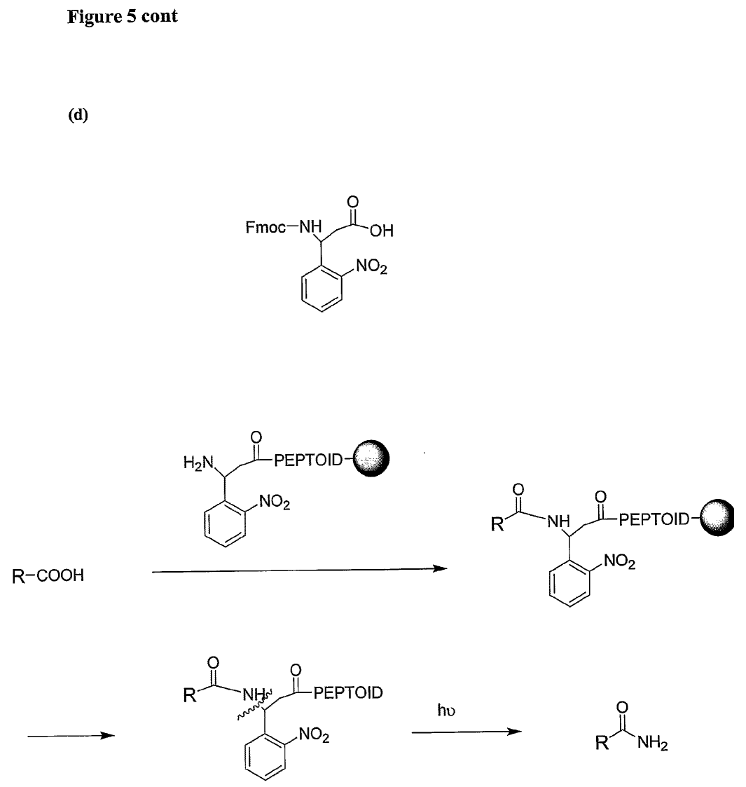

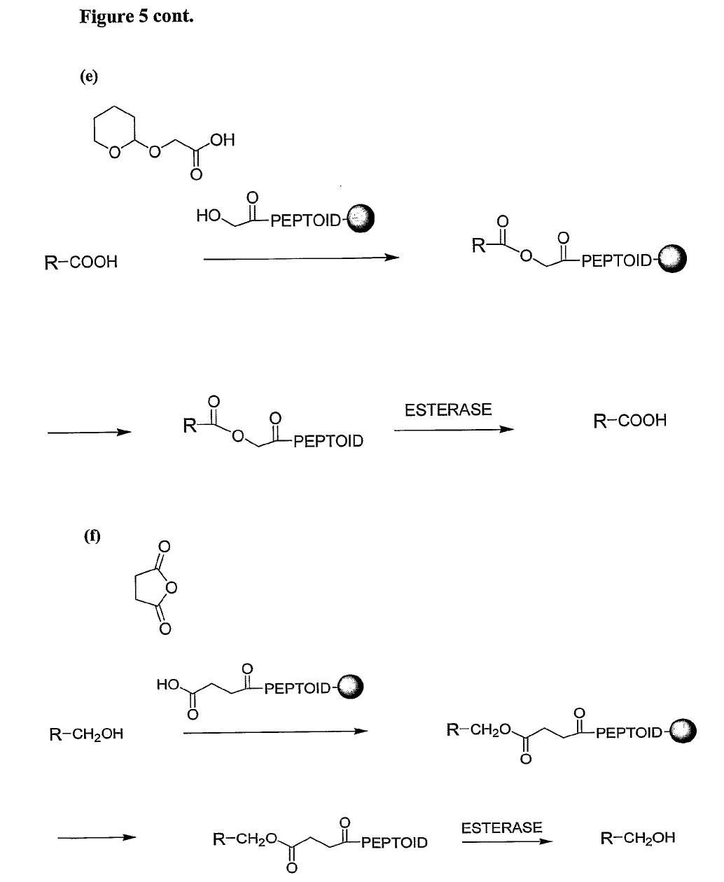

As an alternative to the linkers shown in FIGS. 1 and 3, it is envisaged that linkage of the therapeutic agent to the poly-peptoid carrier, e.g. a PPL, may be via a sulphide linker as shown in FIG. 5b. It is envisaged that this will provide a stable conjugate in skin. However, as indicated above, it may be found desirable to provide the active therapeutic agent linked to the poly-peptoid by a linkage cleavable in skin to release the chosen therapeutic. Such linkage is, for example, of particular interest where the therapeutic is a PNA or an anti-sense polynucleotide designed to target a mRNA. Examples of such linkers are a disulphide linker as shown FIG. 5a or an esterase cleavable linker. Two examples of esterase cleavable linkers are shown in FIGS. 5c and 5d. It is additionally proposed that the therapeutic agent may be linked to the poly-peptoid carrier by a photolytic linker which requires light to release the therapeutic agent. Examples of such linkers have previously been described in Rich and Gurwara, J. Am. Chem. Soc. (1975) 97, 1575 and Brown et al., Mol. Div. (1995) 1, 4 and these are illustrated in FIGS. 5e and f.

### Screening Tests

Poly-peptoid-agent conjugates as discussed above may also be employed in screening tests to identify agents suitable for skin delivery in accordance with the invention to treat a skin characteristic either for therapeutic or cosmetic purpose. Such a method will comprise the steps of:

(i) providing a conjugate in which the agent to be tested is conjugated to the poly-peptoid, preferably a PPL, said conjugate additionally carrying a label detectable in skin, e.g. a fluorescent label such as a fluorescein group label;
(ii) applying said conjugate to the cutaneous surface of a skin sample ex vivo, and
(iii) determining whether the labelled conjugate penetrates into the skin sample. Such a method may further comprise determining whether a characteristic of the skin sample is changed by penetration into the sample of the conjugate. A suitable system for such testing using a diffusion chamber is described in the examples' section. The labelled conjugate may for example be a conjugate of formula IV wherein the number of poly-pseudo-lysine units is 7.

### Cosmetic Uses

As indicated above, poly-peptoid-agent conjugates as discussed above may have cosmetic use which may be exploited independently of any therapeutic use. Thus, the invention provides cosmetic use of a conjugate in which an agent suitable for changing a skin characteristic is conjugated to a poly-peptoid carrier of general formula I, preferably poly-pseudo lysine as discussed above, and said conjugate is applied to skin whereby said conjugate penetrates into the skin and said agent is effective to change said skin characteristic.

Of particular interest for cosmetic use are conjugates as noted above which can enter skin and affect skin pigmentation, notably conjugates in which PPL is conjugated to αMSH or an active derivative thereof which binds to MC1R and PPL conjugates designed to decrease or prevent tyrosinase expression in skin, especially, for example, PNA-PPL conjugates for this purpose. αMSH-PPL conjugates, e.g. αMSH-PPL [98} and αMSH-PPL [99] and variants thereof wherein αMSH is substituted by an active derivative thereof, may be advantageously employed to promote tanning in individuals. This may be particularly favoured in individuals heterozygous for a genetic variant of the MC1R which causes fair skin in the heterozygous state (and which causes red hair and fair skin in the homozygous variant/compound heterozygous variant state). Use of PPL oligomers to deliver αMSH or an active variant thereof into human skin before sun exposure could increase tanning and thereby reduce risk of UV-induced DNA damage in skin and therefore skin cancer development.

The invention also extends to cosmetic preparations suitable for a cosmetic use as discussed above, including for example, creams, ointments, gels and lotions such as creams and lotions including a PNA-PPL conjugate or antisense oligonucleotide-PPL conjugate, e.g. for targeting tyrosinase expression in skin, which might be referred to as ‘gene creams or lotions.’

The following examples illustrate the invention.

### EXAMPLES

### Example 1

### Skin Penetration by Fluorescein-Labelled αMSH-PPL[981 and αMSH-PPL[99]

### Material and Methods

### Compounds

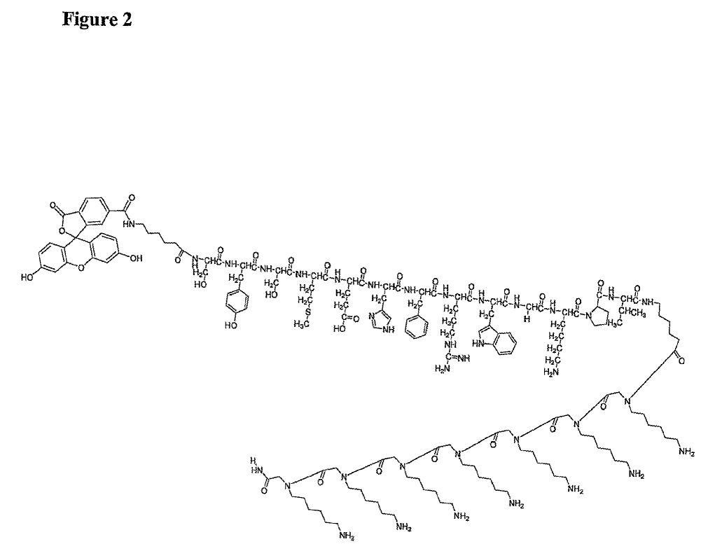

The following compounds were synthesized: αMSH, αMSH conjugated to PPL as shown in FIG. 1 and FIG. 3 (αMSH-PPL[98] and αMSH-PPL[99]), αMSH conjugated to a fluoroscein group (Fluo-MSH), fluorescein-labelled MSH-PPL[98] as shown in FIG. 2 and fluorescein-labelled MSH-PPL[99] as shown in FIG. 4 (hereinafter referred to as fluo-MSH-PPL[98] and MSH-PPL[99]-fluo respectively).

Initially the biological efficacy of the αMSH was tested against that of two commercially available αMSHs (Bachem, UK and Chemicon, UK) in pigmentation assays, and found to be similar (data not shown). For the ligand binding assays, the radiolabelled superpotent analogue ¹²⁵I-Nle⁴-D-Phe⁷-αMSH (¹²⁵I-NDP-MSH) was used.

### Cell Culture

B16G4F, B16F10, S91, A431, HEK293 and HaCaT cells were cultured at 37° C. and 5% CO₂ in Dulbecco's modified Eagles medium (DMEM) with 10% fetal bovine serum (FBS), 2 mM L-glutamine, and (i) 2 μg/ml ciprofloxacin or (ii) 100 U/ml penicillin with 100 μg/ml streptomycin. B16UWT-3 cells (i.e. B16G4F cells which had been stably transfected with wild type MC1R) were cultured in the same medium as the B16G4F line with the addition of geneticin (G418; 1.5 mg/ml). HT1080 cells were grown in Roswell Park Memorial Institute (RPMI) 1640 medium with 10% FBS, 100 U/ml penicillin/100 μg/ml streptomycin and 2 mM L-glutamine and maintained at 37° C. and 5% CO₂. Primary cultures of B lymphocytes were grown in RPMI 1640 medium enriched with L-glutamine, and 5% heat inactivated human AB serum, 1% sodium pyruvate, and 100 U/ml penicillin/100 μg/ml streptomycin.

### Ligand Binding Assay

Competitive ligand binding experiments at the MC1R were carried out using B16UWT-3 cells in duplicate wells on two separate occasions. Forty thousand cells per well were seeded into 96-well plates, and following overnight incubation, the cells were washed once with binding buffer (minimal essential medium with Earle's salts, 25 mM HEPES pH 7.0, 0.2% bovine serum albumin, 1 mM 1,10-phenanthroline, 0.5 mg/l leupeptine and 200 mg/l bacitracin). Cells were incubated for 2 hours at room temperature with 0.05 ml of binding buffer containing 15,000 c.p.m. of ¹²⁵I-NDP-MSH and concentrations of unlabelled ligand ranging from 10⁻²-10⁻³ nM. The cells were subsequently washed twice with 0.2 ml of ice cold binding buffer and detached from the plates with 0.1 ml of 0.1 M NaOH. Radioactivity was counted using a Packard auto-Gamma Counter (Packard Bioscience Ltd., UK) and data analysed with a software package for radioligand binding analyses (GraphPad Software, Inc. San Diego, USA).

### Pigmentation Assay

S91 Cloudman murine melanoma cells were cultured for 5 days in the presence or absence of αMSH compounds. All experiments were carried out in triplicate wells on three separate occasions. Following culture, cells were washed twice with PBS and then dissociated with cell dissociation solution (Sigma, UK). An aliquot of cells were counted using a haemocytometer, and the remaining cells pelleted by centrifugation prior to lysis of the pellet with 200 μl of 1M NaOH. Melanin concentrations were determined against a standard synthetic melanin dose range using a spectrophometric plate reader (emission 450-492 nm).

### Immunosuppression Assay

PBMCs were purified using density gradient (Lymphoprep) centrifugation and resuspended in RPMI 1640 medium (Invitrogen Life Technologies) enriched with L-glutamine and supplemented with 5% heat-inactivated human AB serum (Sigma-Aldrich), 100 U/ml penicillin and 100 μg/ml streptomycin, and 1% sodium pyruvate (Invitrogen Life Technologies). To each well of a 48-well plate (Nunc), 1.4×10⁶ cells were added and incubated in the presence or absence of different concentrations of MSH, MSH-PPL[98], MSH-PPL[99] and Fluo-MSH-PPL[98] (10⁻¹³ M, 10⁻¹² M, 10⁻¹¹ M, 10⁻⁹ M, 10⁻⁸ M, 10⁻⁷ M; Bachem); all cultures were performed in triplicate. Lymphocyte proliferation was stimulated by the addition of the streptococcal Ag mixture streptokinase-streptodornase (SK/SD 0.5/0.125 U/ml; Varidase (Phoenix Pharmaceuticals) or Phytohemagglutinin (PHA, 4 μg/ml; Sigma-Aldrich). Cultures were incubated at 37° C. in 5% CO₂, and on day 6 after challenge with varidase or on day 3 after challenge with PHA, proliferation was assessed in triplicate by [³H]thymidine incorporation.

### Fluorescent Microscopy and Confocal Microscopy

Experiments to investigate the transport of each compound into cells in culture were performed at 3 hours and 24 hours. Cells were seeded (at 1×10⁵ cells per well for 3 hours treatment or 5×10⁴ cells per well for 24 hours treatment) on 1% (w/v) poly-L-lysine coated glass coverslips in 12 well plates and incubated overnight at 5% CO₂ and 37° C.; poly-L-lysine coating of glass coverslips was employed to prevent the fluo-labelled PPL compounds adhering to the glass and causing background fluorescence. Cells were then washed with PBS, and incubated at 5% CO₂ and 37° C. with the compound in the medium for 3 hours or 24 hours. The medium was subsequently removed and the cells washed twice with PBS, prior to fixation with 4% paraformaldehyde for 7 minutes, and then fixation was stopped with 50 mM ammonium chloride for 10 minutes. Cells were subsequently washed twice with PBS, and coverslips were mounted (with the cells facing downwards) onto a glass slide with vectashield, and the edges sealed with clear nail varnish to prevent drying. Fluorescence was detected by fluorescence microscopy and/or confocal microscopy. For experiments examining the half-life of the relevant compound inside the cells, following the removal of the medium containing the compound, the coverslips were washed twice with PBS and transferred to another clean well with fresh medium for the desired time scale.

### Cytotoxicity Assay

Cells were plated at 2×10⁴ cells in 100 μl medium in 96 wells plate and incubated overnight at 37° C. and 5% CO₂. Cells were then washed with PBS to remove lactate dehydrogenase (LDH) released during the overnight incubation, and 200 μl of fresh medium with 1% serum and the relevant compound under investigation added to each well. Appropriate controls were included in each experiment, e.g. the assay medium with no cells, no drug/compound control, and equal volumes of 1% Triton X and cells (as high control). All experiments were carried out in triplicate wells on three separate occasions. Following incubation with the test compound, 100 μl of supernatant from each well was transferred into respective wells of an optically cleared flat bottom 96 well plate. 100 μl of LDH reaction mixture (freshly made before use) was added to each well and incubated for 30 minutes at room temperature (15-25° C.) in the dark. The resulting colour was quantified using a plate reader at 490-492 μm, and the percentage cytotoxicity calculated as follows:

$$\% cytotoxicity = \frac{Experimental value - low control}{High control - low control} \times 100$$

### Fluorescence Activated Cell Sorting (FACS) Analysis

Cells were seeded at 1×10⁵ cells per well in 6 well plates and incubated overnight at 37° C. with 5% CO₂ prior to incubation with the compounds under investigation in the relevant culture medium for 3 hours and 24 hours. Cells were subsequently washed twice with PBS and dissociated with cell dissociation solution (Sigma, UK). Following the addition of fresh medium, cell suspensions were centrifuged at 1200 rpm for 5 mins. The cell pellets were washed in PBS, re-centrifuged and re-suspended in 300 μl of FACS buffer and the fluorescence quantified using a FACS-Scan fluorescence activated cell sorter.

### Synthesis of αMSH and Fluorescein-Labelled αMSH

Alpha-MSH (Ac-SYSMEHFRWGKPV-NH₂) and fluorescein labelled α-MSH (Fluo-Ahx-SYSMEHFRWGKPV-NH₂), used as references for the test of αMSH-PPL conjugate (PPL oligomer, n=7), were prepared by solid phase synthesis.

The α-MSH peptide was assembled on polystyrene resin functionalised with Rink amide linker.

### Resin Preparation

Fmoc protected Rink linker (0.376 g, 0.6975 mmol) was mixed with HOBt (0.094 g, 0.6975 mmol) in DMF (3 ml) and stirred for 10 min. Then DIC (0.109 mL, 0.6975 mmol) was added to the mixture and stirred for other 10 min. The solution was added to 250 mg of aminomethyl resin (loading 0.93 mmol/g) pre-swollen in 5 ml of DCM for 20 min and then drained. The mixture was stirred on a rotary shaker for 14 hrs. After the solvent was removed and the resin washed with DMF, DCM, MeOH, Et₂O (3×10 ml for each solvent). Completion of the reaction was monitored using a qualitative Ninhydrin test.

### Peptide Synthesis

The fmoc-Rink linker functionalised resin was swollen for 20 mins in DMF and then drained. The fmoc protecting group was removed by adding 3 ml of piperidine solution (20% in DMF) and stirring the mixture for 20 mins. The resin was washed with DMF, DCM, MeOH, Et₂O (3×10 ml for each solvent), all repeated 2 times. Complete de-protection was monitored using a qualitative Ninhydrin test. The fmoc-protected amino acids (starting from V) (0.6975 mmol, 3 eq.) were dissolved in 3 ml of DMF and stirred with HOBt (0.6975 mmol) for 10 mins. Then DIC was added (0.6975 mmol) and the solution stirred for other 10 mins. The solution was added to the resin (pre-swollen in DMF for 20 mins and drained) and the mixture stirred on a rotary shaker at room temperature for 3 hrs. Coupling completion was monitored using a qualitative Ninhydrin test. Fmoc de-protection and amino acid coupling was repeated to obtain the desired sequence. Proline de-protection and consecutive coupling was monitored using the Chloranil test.

Di-protected amino acids used: Fmoc-Lys (Boc)-OH, Fmoc-Trp(Boc)-OH, Fmoc-Arg (Pbf)-OH, Fmoc-His(1-Trt)-OH, Fmoc-Glu (OtBu)-OH, Fmoc-Ser(tBu)-OH, Fmoc-Tyr (tBu)-OH.

After the coupling of the last amino acid, the resin was split into two parts for further processing as in 1 and 2 below

1. N-Acylation of last residue: after fmoc de-protection the resin (pre-swollen in DMF) was treated with 2 ml of AC₂O/Pyr (1/1) solution. The mixture was stirred using a rotary shaker for 4 h. Reaction completion was monitored using a qualitative Ninhydrin test.

2. Fluorescein Labelling:

Spacer: fmoc-6-aminohexanoic acid was coupled using the same method described for the other amino acids.

Fluorescein coupling: 5(6) carboxyfluorescein (3 eq) was dissolved with PyBop (2.8 eq) in DMF to a concentration of 0.1 M. DIPEA (6 eq) was added to the solution then added to the pre-swollen resin. The mixture was stirred over night using a rotary shaker. Reaction completion was monitored using a qualitative Ninhydrin test.

α-MSH and Fluorescein-Labelled α-MSH TFA Cleavage and Ether Precipitation

TFA solution: 81.5% TFA, 5% thioanisole, 5% phenol, 5% water, 2.5% EDT(1,2 ethanedithiol), 1% TIS (triisopropylsilane).

TFA solution (2 ml) was added to the dry resin. The mixture was stirred at room temperature for 4 hrs using a rotary shaker. The mixture was filtered and the resin washed with another 2 ml of TFA solution.

TFA was removed under vacuum. Cold ethyl ether (4 ml) was added to the residue, then triturated to obtain a free suspension. The suspension was transferred into a centrifuge tube containing 10 ml of cold ethyl ether. The peptide was precipitated by centrifugation. The peptide was suspended and precipitated in ether 3 times. α-MSH and Fluorescein-labelled α-MSH were purified by semiprep-HPLC.

α-MSH: MS (ES⁺) 556.0 (M+3H)³⁺, 833.3 (M+2H)²⁺, 1687.4 (M+Na)⁺

Fluo-α-MSH: MS (MALDI-TOF): 2094.8 (M)

### Synthesis of Fluorescein-Labelled αMSH-PPL[98]

Synthesis of the PPL oligomer (n=7) was carried out as described in Peretto et al. Chem. Commun. (2003) 2312-2313 starting with a base monomer (VI) prepared starting from hexane-1,6-diamine:

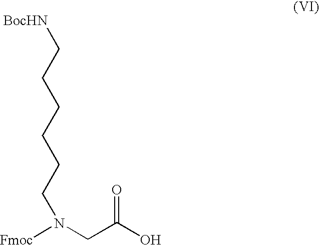

The PPL was constructed from this monomer on the Fmoc-protected side of the lysine-type side chain using an Fmoc strategy. The assembly was on PEGA resin using a Rink amide linker. Fmoc-Lys (Dde)-OH (VII) was bound to the resin.

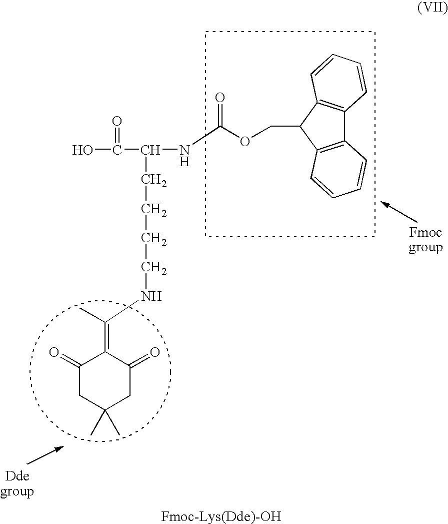

### General Procedure for Peptoid Coupling

The peptoids were assembled on Rink PEGA resin (200 mg) functionalised with Fmoc-Lys (Dde) at a loading of 0.4 mmol/g. The first step was the Fmoc-deprotection of the supported lysine by treating the resin with a solution of 20% piperidine in DMF (5 ml, 2×10 minutes). In each of the coupling steps, the Fmoc-protected monomer and PyBroP in a 2-fold molar excess were added to the resin (previously swollen in DCM for 20 min.), for a final concentration of 0.08M in DCM. Diisopropylethylamine (4 eq.) was added, and the mixture was stirred on a rotary shaker for 4 hrs. The resin was then drained and washed sequentially with DCM, DMF, MeOH, diethyl ether (15 ml each solvent). Deprotection of the Fmoc group was achieved by treating the resin with a solution of 20% piperidine in DMF (5 ml 2×10 minutes). These two steps (coupling and Fmoc deprotection) were repeated until an oligomer of 7 residues was built up leaving the last monomer Fmoc protected. A small amount of compound cleaved from the support was analysed by MALDI-TOF MS and the spectra obtained showed the desired compound (VIII)

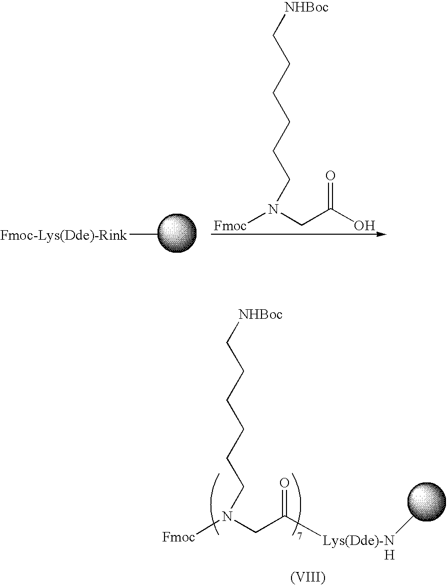

### Peptoid Cleavage

The resin was suspended in TFA/DCM (95/5) 10 ml/g and left to stir for 30 minutes. After filtration the resin was washed with TFA/DCM (95/5, 10 ml/g) The solvent was evaporated under a nitrogen flow and the PPL precipitated with ether.

### Coupling of Fluorescein-Labelled αMSH: see Peptide Synthesis Above.

### Microwave-Assisted Synthesis of Fluorescein-Labelled αMSH-PPL[98]

Mild thermal effects using microwave heating have proven to be successful in enabling rapid and efficient couplings to generate the hepta peptoid and conjugate it to αMSH. Details of the protocol are given in Fara et al. Tetrahedron letters (2006) 47, 1011-1014.

### Synthesis of Fluorescein-Labelled αMSH-PPL [99]

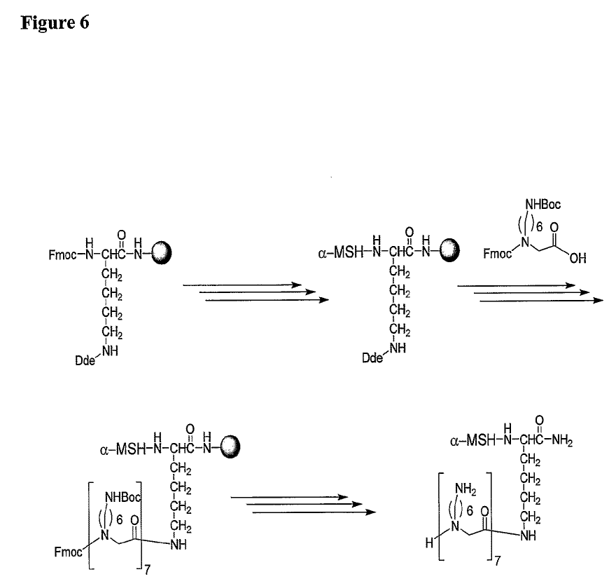

Synthesis was carried out on resin according to the scheme given in FIG. 6.

### Resin Preparation:

Fmoc protected Rink linker (3 eq) were mixed with HOBt (3 eq) in DMF (to a concentration 0.2 M) and stirred for 10 min. Then DIC (3 eq) was added to the mixture and stirred for other 10 min. The solution was added to PEGA resin (1 eq) pre-swollen in DCM for 20 min and then drained. The mixture was stirred for 14 hrs. After the solvent was removed and the resin washed with DMF, DCM, MeOH (3×10 ml for each solvent). Reaction was monitored using the Kaiser test.

### Peptide Synthesis:

The fmoc-Rink linker functionalised resin was swollen for 20 min in DMF, and then drained. The fmoc protecting group was removed by adding a solution of piperidine (20% in DMF) and stirring the mixture for 20 min. The resin was washed with DMF, DCM, MeOH (3×10 ml for each solvent), all repeated 2 times.

Coupling of Fmoc-Lys(Dde)-OH: the amino acid (3 eq) was dissolved in DMF (0.2 M) and stirred with HOBt (3 eq) for 10 mins. Then was added DIC (3 eq) and the solution stirred for other 10 min. The solution was added to the resin (pre-swollen in DMF for 20 min. and drained) and the mixture stirred at room temperature for 3 hrs.

Synthesis of α-MSH on the Fmoc protected side of Lysine: the α-MSH was prepared on the Fmoc protected side of lysine. Fmoc de-protection and amino acid coupling (as described above starting from V) were repeated until the desired sequence was obtained. Proline de-protection and consecutive coupling was monitored using the Chloranil test. Fluorescein labelling: This was carried out as above using a 6-aminohexanoic spacer and 5(6)-carboxyfluorescein.

### Synthesis of the Peptoid on the Dde Protected Side of the First Lysine

Dde-protecting group was removed by treating the resin with a 20% solution of NH2OH.HCl/imidazole (1/0.75 eq) in NMP/DMF (5/1) for 1 h. After the resin was filtered and washed with DMF, DCM, MeOH (3×15 ml each solvent). In each of the coupling steps, the Fmoc-protected monomer and PyBrop in a 3-fold molar excess were added to the resin (previously swollen in DCM for 20 min.), for a final concentration of 0.2 M in DCM. Diisopropylethylamine (6 eq.) was added, and the mixture was stirred for 4 hrs. The resin was then drained and washed sequentially with DCM, DMF, MeOH (3×15 ml each solvent). Reaction was monitored using the Chloranil test. Deprotection of the Fmoc group was achieved by treating the resin with a solution of 20% piperidine in DMF (2×10 minutes). These two steps (coupling and Fmoc deprotection) were repeated until an oligomer of 7 residues was obtained. The final Fmoc group was removed before cleavage.

Cleavage: TFA solution: 81.5% TFA, 5% thioanisole, 5% phenol, 5% water, 2.5% EDT(1,2 ethanedithiol), 1% TIS (triisopropylsilane).

TFA solution was added to the resin as above. The mixture was stirred at room temperature for 4 hrs. After concentrating the solution in vacuo the suspension was transferred into a centrifuge tube containing cold ethyl ether and the peptide was precipitated by centrifugation. The peptide was suspended and precipitated in ether 3 times.

### Skin Penetration Studies

The penetration of the relevant compounds into mouse, pig and human skin was investigated using a Franz diffusion chamber system; this approach ensured a sealed system which prevented any leakage between the upper donor compartment and the lower receptor compartment. Circular full thickness skin samples (6 mm diameter) which had been cut with a punch biopsy blade were mounted into the diffusion chamber, with the stratum corneum facing upwards so that it was exposed to donor compartment, and with the receptor compartment filled with PBS. The compound under investigation was applied to the donor compartment so that it lay on the outer skin surface, and the chamber incubated at room temperature or at 37° C. for up to 3 hours. At the end of this incubation period, the remaining solution was removed from the upper compartment, and the skin washed three times with PBS. The skin was then bisected and half of it was snap frozen in liquid nitrogen for cryosectioning whereas the other half was fixed in 4% paraformaldehyde for 3 hours prior to paraffin blocking for cutting tissue sections. The skin sections were mounted on glass and visualised under fluorescent microscopy.

### Results

### αMSH Covalently Bound to PPL Retains its Activity.

Initial experiments sought to determine whether αMSH retained its biological activity when bound to PPL. Previous research had shown that αMSH stimulates pigmentation of melanocytes and melanoma cells in vitro via increasing intracellular cyclic-AMP at concentrations of 10⁻⁸ M to 10⁻⁶ M. Pigmentation assays demonstrated that MSH-PPL[98] and MSH-PPL[99], as well as Fluo-MSH, Fluo-MSH-PPL[98] and MSH-PPL[99]-Fluo stimulated melanin synthesis by melanoma cells with similar potency to αMSH at 10⁻⁸ M and 10⁻⁶ M.

As noted above, recent work has also identified that αMSH has immunomodulatory activity and can suppress lymphocyte proliferation in vitro at 10⁻¹³ M to 10⁻¹² M; although up to 80% inhibition of streptokinase/streptodornase induced lymphocyte responses can be observed, the average suppression is 20% to 25% with this compound [Cooper et al., J. Immunol 2005; 175: 4806-13]. MSH-PPL[98] and MSH-PPL[99] each suppressed phytohaemagglutin-induced lymphocyte proliferation similar to that expected of αMSH.

It is known that both the effects of αMSH on pigmentation and some of its immunosuppressive activities occur via binding of the peptide to the melanocortin 1 receptor (MC1R). The binding affinity of MSH-PPL[98] for MC1R was investigated and was seen to exhibit a similar affinity to that of αMSH; consistent with the greater potency of NDP-MSH in stimulating cyclic-AMP through activation of MC1R, NDP-MSH demonstrated a greater affinity for MC1R in this assay.

PPL Transports αMSH into Cells

To examine whether αMSH is transported into cells by PPL, fluorescently-tagged compounds were employed and intracellular fluorescence documented as a measure of penetration. Fluo-MSH did not penetrate into any of the cell types used in these experiments. However, Fluo-MSH-PPL[98] and MSH-PPL[99]-Fluo at 10⁻⁵ M penetrated in large amounts into all cell types tested (B16G4F, B16UWT-3, HEK-293, and B-lymphocytes) Fluorescence microscopy showed bright green fluorescent cells following incubation with 10⁻⁵ M Fluo-MSH-PPL[98] and 10⁻⁵ M MSH-PPL[99]-Fluo, suggesting that these compounds had either adhered to the cell membrane or entered into the cell. Confocal microscopy confirmed that the PPL conjugated compounds had penetrated into the cells, and that uptake of Fluo-MSH-PPL[98] and MSH-PPL[99]-Fluo had occurred in 95-100% of cells in each culture.

PPL-αMSH Compounds do not Exhibit Greater Toxicity than the Non-PPL αMSH Compounds

In the above cell culture experiments, there was no evidence that the PPL conjugated compounds were toxic as assessed by cell morphology and cell viability. In addition, LDH assays showed no increase in cytotoxicity by the Fluo-PPL and PPL-αMSH compounds over that seen with αMSH alone, indicating that the PPL carrier was not toxic in this assay.

PPL Allows αMSH to Penetrate into Skin

Penetration of αMSH into skin was examined using an ex-vivo mouse, pig and human skin model. Fresh skin was taken separately from the dorsum of euthanased mice, the ear of euthanased pigs, and by excision during surgical procedures on dermatology patients. The skin samples were inserted into tightly-sealed diffusion chambers and incubated with the compounds under study as described above. To enable visualisation of αMSH and related molecules in these experiments, fluorescein-labelled compounds were employed. αMSH-PPL[99]-Fluo applied at a concentration of 10⁻³ M penetrated in large amounts into mouse, pig and human skin on multiple occasions; by contrast, in multiple experiments fluorescein-labelled-αMSH did not penetrate into skin (except on a single occasion in mouse skin). Interestingly, Fluo-αMSH-PPL[98] did not penetrate into skin to the same extent as αMSH-PPL[99]-Fluo.

### Example 2

### TyrPNA and TyrASO Covalently Bound to PPL Inhibit Tyrosinase Activity and Pigmentation

### Compounds

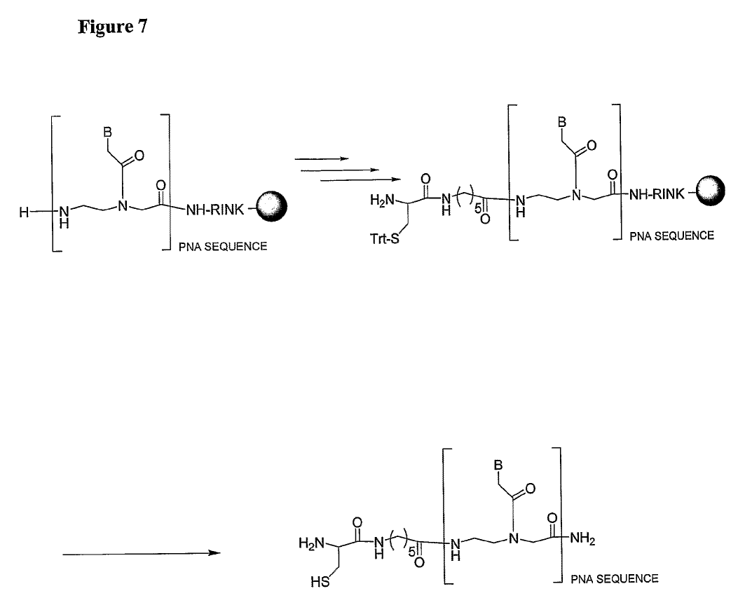

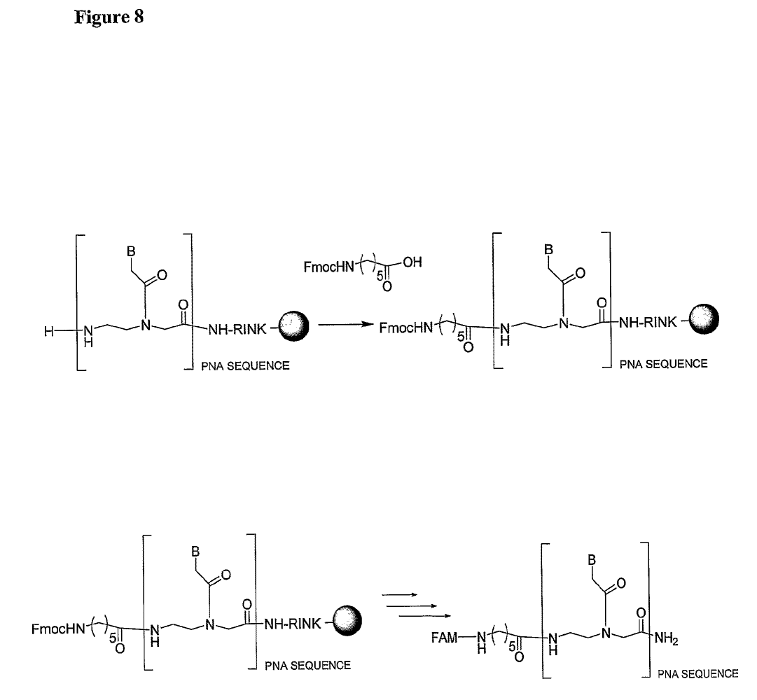

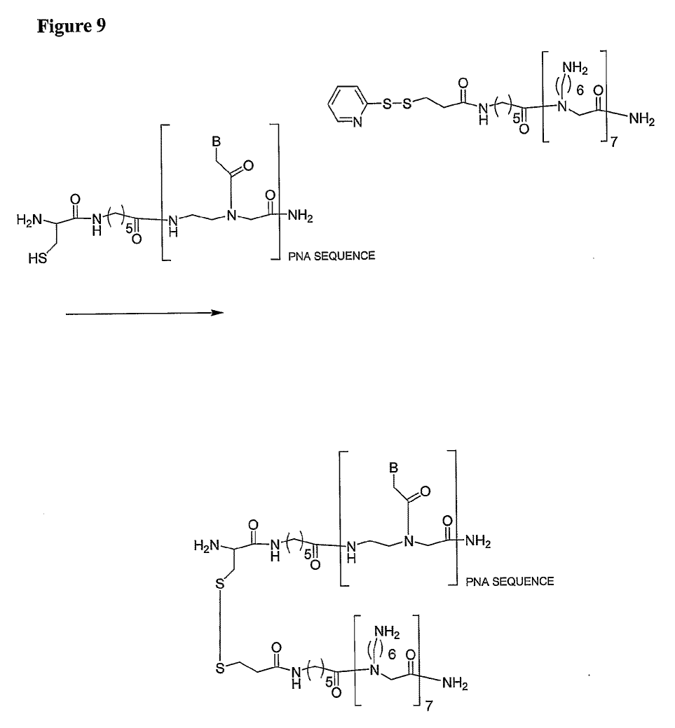

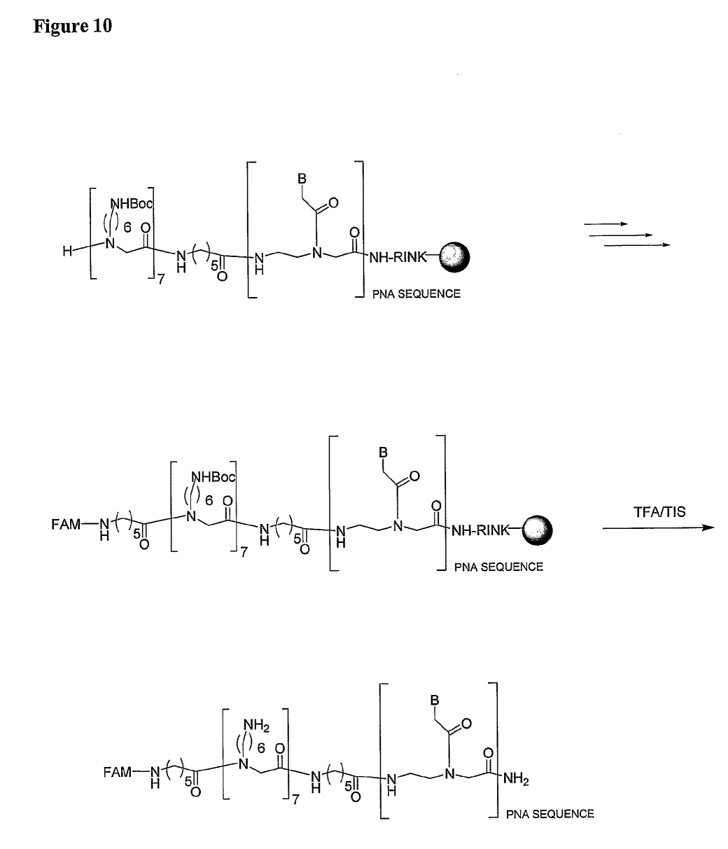

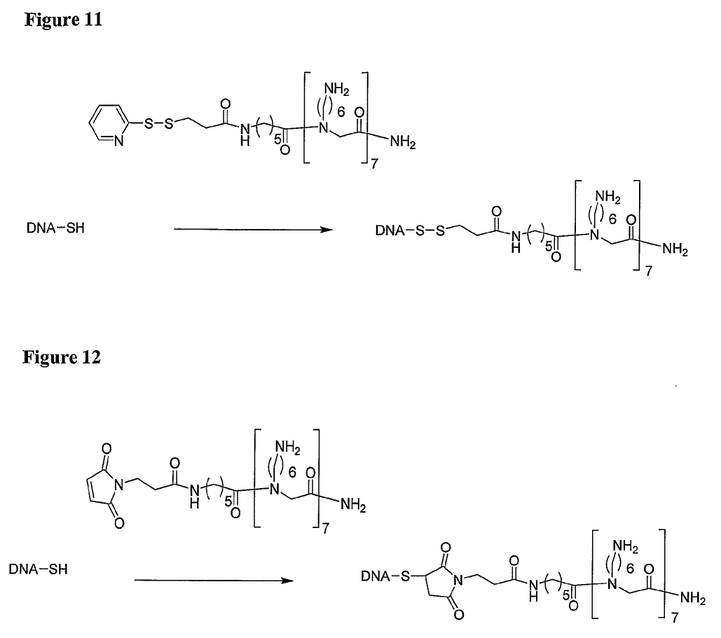

The following compounds were used: a 15 mer antisense peptide nucleic acid (GAA CAT TTT CTC CTT; SEQ. ID no. 1) targeting murine tyrosinase and modified as shown in FIG. 7 (the modified PNA is hereinafter referred to as TyrPNA) and a 19 mer antisense oligonucleotide, also targeting murine tyrosinase (TyrASO; purchased from Sigma), and each of these agents conjugated to 7 mer PPL (referred to as TyrPNA-PPL and TyrASO-PPL respectively). In the TyrPNA-PPL conjugate tested, the PPL was conjugated to the PNA using a linker containing a disulphide bridge as shown in FIG. 9. Two different antisense oligonucleotide-PPL conjugates were assessed for ability to suppress melanin in αMSH stimulated BF1610 cells (for conjugate structures see FIGS. 11 and 12). In preliminary experiments, the antisense conjugate with the disulphide bridge appeared to be more effective and is referred to as TyrASO-PPL in the results discussed below. In addition, fluorescein-labelled TyrPNA and fluorescein-labelled TyrPNA-PPL (hereinafter referred to as TyrPNA-fluo and TyrPNA-PPL-fluo respectively) were employed with the structures shown in FIGS. 8 and 10 respectively.

### Synthesis of TyrPNA, TyrPNA-PPL, TyrPNA-Fluo TyrPNA-PPL-Fluo

All the compounds were synthesized on a Rink functionalized PEGA resin. The Construction of the desired PNA was prepared as published in Bialy et al., Tetrahedron (2005) 61, 8295-8305; “Dde-protected PNA monomers, orthogonal to Fmoc, for the synthesis of PNA-peptide conjugates.”

General-coupling methods: Fmoc-6-aminohexanoic acid or 5(6)-carboxyfluorescein (5 eq, 0.2 M), HOBt (5 eq) and DIC (5 eq) in DMF were stirred for 5 minutes and added to the resin (previously swollen in DMF for 20 min.), and the mixture was stirred for 3 hrs. The resin was then drained and washed with DMF, DCM. Coupling completion was monitored by the Kaiser test. In the case of 5(6)-carboxyfluorescein coupling, the resin was then treated with piperidine (20% in DMF, 2×1 h) to cleave the carboxyfluorescein-carboxyfluorescein esters.

Fmoc groups were removed using a solution of piperidine (20% in DMF, 2×15 min).

Resin Cleavage: TFA (95%), TIS (2.5%), water (2.5%). Compounds were precipitated and washed with cold ether.

Peptoid Synthesis Monomer (5 eq, 0.2 M) HOBt (5 eq) and DIC (5 eq) in DMF were stirred for 5 minutes and added to the resin (previously swollen in DMF for 20 min.), and the mixture was stirred for 4 h. The resin was then drained and washed with DMF, DCM. Coupling was monitored by Chloranil test. Following Fmoc deprotection, the reaction was repeated to give the desired length peptoid. Before cleavage from the resin the terminal Fmoc group of the 7-mer peptoid was removed.

Synthesis of TyrPNA and Tyr-PNA-fluo (see FIGS. 7 and 8): Fmoc-6-aminohexanoic acid, Fmoc-Cys(Trt)-OH and 5(6)-carboxyfluorescein were coupled as follows: The acid (5 eq, 0.2 M), HOBt (5 eq) and DIC (5 eq) in DMF were stirred for 5 minutes and added to the resin (previously swollen in DMF for 20 min.), and the mixture was stirred for 3 hrs in accordance with the general coupling procedure given above. The resin was then drained and washed with DMF, DCM. Coupling completion was monitored by the Kaiser test. The Fmoc group was removed as also described above. For fluorescein labelling, after the coupling of 5(6)-carboxyfluorescein the resin was treated with piperidine (20% in DMF, 2×1 h) to cleave any dye-dimers. Cleavage of compounds from the linker-resin was achieved as additionally described above and the compounds were precipitated and washed with cold ether.

Synthesis of TyrPNA-SS-PPL (see FIG. 9):

PNA-SH (TyrPNA) and the Ar-SS-Peptoid (1 equivalent) were stirred in water over-night to afford the PNA-SS-Peptoid conjugate.

Synthesis of TyrPNA-PPL-fluo was carried out in accordance with the general procedures given above as shown in FIG. 10.

### Synthesis of TyrASO-PPL Conjugates

Synthesis of TyrASO-PPL conjugates was carried out according to the schemes shown in FIGS. 11 and 12. DNA-SH (5′-thiol-DNA oligo) and the Ar-SS-Peptoid (1 equivalent) in water were stirred overnight to afford the TyrASO-SS-PPL conjugate. DNA-SH and the malemide-Peptoid (1 equivalent) in water were stirred over night to afford the TyrASO-S-PPL conjugate. As noted above, the TyrASO-SS-PPL conjugate is referred to further below.

### Cell Culture And Pigmentation Assay

B16F10 murine melanoma cells were cultured for 48 hours in the presence or absence of αMSH (10⁻¹⁰ M) with or without TyrPNA or TyrASO and separately with or without TyrPNA-PPL or TyrASO-PPL. Pigmentation was assessed as described above.

### Tyrosinase Assay

Following culture of B16F10 murine melanoma cells as for the pigmentation assays, cells in 96 well plates were washed twice with PBS and then lysed with 20 μl of 50 mM sodium phosphate buffer (Na₂HPO₄+NaH₂PO₄, ph 6.9) containing 0.05% Triton-X-100. For the measurement of tyrosinase activity, 180 μl of freshly prepared reaction mixture (6.3 mM MBTH, 1.1 mM L-DOPA in 48n1M sodium phosphate buffer at pH 7.1) containing 4% v/v N,N-dimethylformamide was added. The kinetic study was performed spectrophotometrically at absorbance 508 nm in mOD/min over 60 min at a constant temperature of 37° C. with comparison against standard mushroom tyrosinase. The end point reading was also taken after 1 hour of the reaction at absorbance 508 nm.

### TyrPNA and TyrASO Covalently Bound to PPL Inhibits Tyrosinase Activity and Pigmentation

The B16F10 cell line is pigmented, but when cultured in 12 well plates the cell pellets following dissociation and centrifugation are very lightly coloured beige/cream. However, the addition of αMSH (10⁻¹⁰ M) to the cultures alters the colour of the cell pellets to brown/black. Addition of TyrPNA-PPL (10⁻⁵ M) in the presence of αMSH inhibited the darkening of the cell pellet whereas addition of TyrPNA (10⁻⁵ M) with αMSH did not prevent the increased production of melanin by the cells. Quantification of melanin by spectrophotometry confirmed inhibition of pigmentation following the addition of αMSH by TyrPNA-PPL (10⁻⁵ M) but not by TyrPNA (10⁻⁵ M). Consistent with these results, αMSH-induced tyrosinase activity was reduced by TyrPNA-PPL (10⁻⁵ M) and separately TyrASO-PPL (10⁻⁵ M) but not by TyrPNA (10⁻⁵ M) nor by TyrASO (10⁻⁵ M). In addition, TyrASO-PPL (10⁻⁵ M) suppressed basal tyrosinase activity whereas TyrASO (10⁻⁵ M) did not.

PPL Transports PNA into Cells

Fluorescently-tagged compounds were used as noted above and intracellular fluorescence recorded as a measure of penetration. TyrPNA-fluo did not penetrate into B16F10 cells, but TyrPNA-PPL-fluo at 3×10⁻⁵ M and at 10⁻⁴ M penetrated in large amounts into these cells. Fluorescence microscopy showed bright green fluorescent cells following incubation and confocal microscopy confirmed that the PPL conjugated compound had penetrated into the cells, with the intensity of fluorescence suggesting that more TyrPNA-PPL-fluo penetrated at 10⁻⁴ M, and that uptake of TyrPNA-PPL-fluo had occurred in 95-100% of cells in each culture.

Similar results have been obtained when the 15 mer PNA is replaced in the TyrPNA-PPL-fluo conjugate by a shortened 12 mer or 9 mer PNA omitting 5′ terminal bases.

PPL Allows PNA to Penetrate into Skin

Penetration of the 15 mer and 12 mer PNA into human skin was examined as for the penetration of αMSH into skin (see above). TyrPNA-PPL-fluo applied at a concentration of 10⁻³ M penetrated in large amounts into human skin but fluorescein-labelled-TyrPNA alone did not penetrate into skin. Interestingly, while the 15 mer PNA conjugated to PPL was observed in both epidermis and dermis, the 12 mer PNA conjugated to PPL showed more preferential accumulation in the dermis which may be favoured for clinical or therapeutic application. Hence, there is also particular interest in the 12 mer and the equivalent PNA sequence complementary to human tyrosinase coding sequence.

1. A method for delivering a drug into skin, comprising:
topically applying the drug to the skin, wherein the drug is conjugated to a poly-peptoid having at least 3 units of formula I 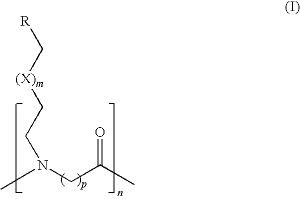
wherein:
p=1 or more, preferably 1, 2 or 4;
X=either CH₂, in which case m=1 or more, e.g. 1 to 5, preferably 3 such that the R group is attached by a hexane side chain; or
CH₂CH═CH and m=1; or
CH═CH and m=1; or
CH₂CC and m=1; or
CC and m=1; and
R=a group selected from primary amine (NH₂), guanidinium (NHC(═NH)—NH₂), amidine (C(═NH)NH₂),
a secondary amine (NHA, where A=alkyl, e.g. Me, Et, nPr, Bn, preferably lower alkyl, e.g. alkyl 1-4, most preferably Me or Et),
a tertiary amine (NA¹A², where A¹ and A² may be the same or different and are alkyl, e.g. Me, Et, nPr, Bn, preferably Me or Et), or
a quaternary amine (N⁺A¹A²A³ wherein each of A¹, A² and A³ may be the same or different and are alkyl, e.g. Me, Et, nPr, Bn, preferably Me or Et).

2. The method of claim 1 in which the drug is conjugated to poly-pseudo-lysine (PPL) having at least 3 units of formula Ia 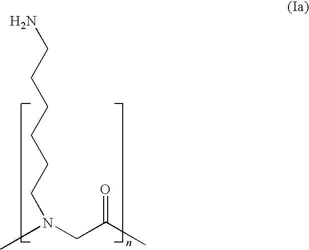
in the manufacture of a therapeutic composition for use in delivering said drug into skin by topical application.

3. The method of claim 1 wherein the therapeutic composition is applied to skin to treat a skin disorder.

4. The method of claim 1 wherein said drug is selected from the group consisting of a polypeptide hormone, a peptide, an enzyme, a PNA, polynucleotide or other drug used to treat a skin disorder.

5. The method of claim 1 wherein the poly-peptoid is poly-pseudo-lysine consisting of 7 monomers of formula Ia.

6. The method of 1 wherein said conjugate is of general formula II

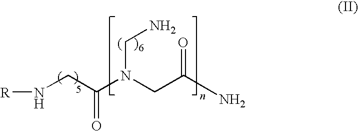

7. The method of claim 6 wherein the drug R is αMSH or an active derivative thereof and n=7 in accordance with formula III

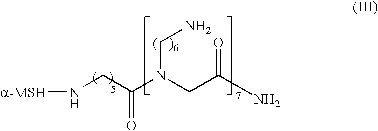

8. The method of claim 1 wherein said conjugate is of general formula IV

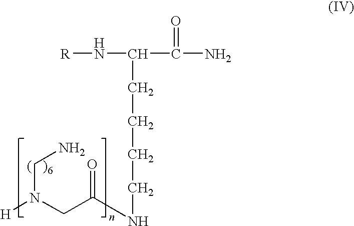

9. The method of claim 8 wherein the drug R is αMSH or an active derivative thereof and n=7 in accordance with formula V

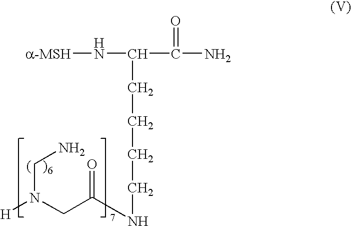

10. A therapeutic composition for use in delivering a drug conjugate into skin by topical application in which the drug of said drug conjugate is conjugated to a poly-peptoid having at least 3 units of formula I 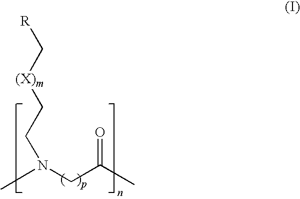
wherein:
p=1 or more, preferably 1, 2 or 4;
X=either CH₂, in which case m=1 or more, e.g. 1 to 5, preferably 3 such that the R group is attached by a hexane side chain; or
CH₂CH═CH and m=1; or
CH═CH and m=1; or
CH₂CC and m=1; or
CC and m=1; and
R=a group selected from primary amine (NH₂), guanidinium (NHC(═NH)—NH₂), amidine (C(═NH)NH₂₁
a secondary amine (NHA, where A=alkyl, e.g. Me, Et, nPr, Bn, preferably lower alkyl, e.g. alkyl 1-4, most preferably Me or Et),
a tertiary amine (NA¹A², where A¹ and A² may be the same or different and are alkyl, e.g. Me, Et, nPr, Bn, preferably Me or Et), or
a quaternary amine (N⁺A¹A²A³ wherein each of A¹, A² and A³ may be the same or different and are alkyl, e.g. Me, Et, nPr, Bn, preferably Me or Et).

11. A therapeutic composition as claimed in claim 10 in the form of a patch or plaster for application to skin and incorporating said drug conjugate in a form such that it will be released into skin.

12. (canceled)

13. A method of delivering an agent into skin to change a skin characteristic wherein said agent is conjugated to a poly-peptoid having at least 3 units of formula I 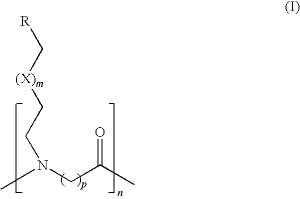
wherein:
p=1 or more, preferably 1, 2 or 4;
X=either CH₂, in which case m=1 or more, e.g. 1 to 5, preferably 3 such that the R group is attached by a hexane side chain; or
CH₂CH═CH and m=1; or
CH═CH and m=1; or
CH₂CC and m=1; or
CC and m=1; and
R=a group selected from primary amine (NH₂), guanidinium (NHC(═NH)—NH₂), amidine (C(═NH)NH₂),
a secondary amine (NHA, where A=alkyl, e.g. Me, Et, nPr, Bn, preferably lower alkyl, e.g. alkyl 1-4, most preferably Me or Et),
a tertiary amine (NA¹A², where A¹ and A² may be the same or different and are alkyl, e.g. Me, Et, nPr, Bn, preferably Me or Et), or
a quaternary amine (N⁺A¹A²A³ wherein each of A¹, A² and A³ may be the same or different and are alkyl, e.g. Me. Et, nPr, Bn, preferably Me or Et)
and said conjugate is applied to skin whereby said conjugate penetrates into the skin and said agent is effective to change said skin characteristic.

14. The method of claim 13 in which an agent suitable for changing a skin characteristic is conjugated to poly-pseudo-lysine having at least 3 units of formula Ia 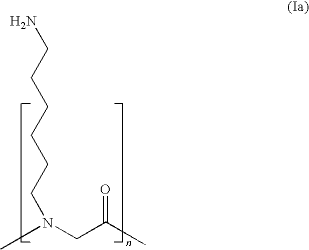
and said conjugate is applied to skin whereby said conjugate penetrates into the skin and said agent is effective to change said skin characteristic.

15. The method of claim 13 wherein the skin characteristic changed is degree of pigmentation.

16. The method of claim 13 wherein the poly-peptoid employed is poly-pseudo-lysine consisting of 7 monomers of formula Ia.

17. The method of claim 13 wherein said conjugate is of general formula II

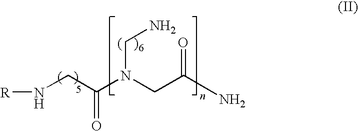

18. The method of claim 17 wherein the agent is αMSH or a derivative thereof which binds the melancortin 1 receptor (MC1R) and n=7 in accordance with formula III

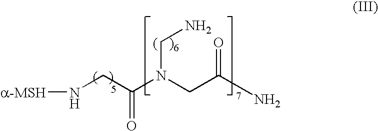

19. The method of claim 13 wherein said conjugate is of general formula IV

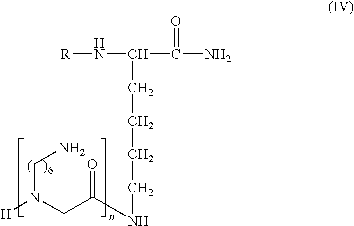

20. The method of claim 19 wherein the agent is αMSH or a derivative thereof which binds the MC1R and n=7 in accordance with formula V

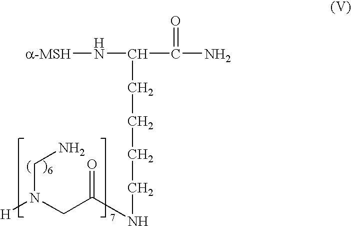

21. The method of claim 15 wherein said agent is a PNA or antisense oligonucleotide which is capable of reducing tyrosinase expression in skin

22. The method of claim 21 wherein said PNA is in a conjugate of general formula VI

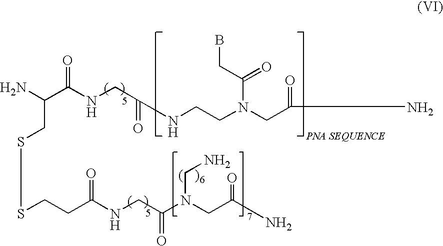

23. A cosmetic preparation for delivering an agent into skin to change a skin characteristic wherein said agent is conjugated to a poly-peptoid having at least 3 units of formula I 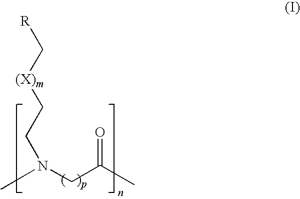
wherein:
p=1 or more, preferably 1, 2 or 4;
X=either CH₂, in which case m=1 or more, e.g. 1 to 5, preferably 3 such that the R group is attached by a hexane side chain; or
CH₂CH═CH and m=1; or
CH═CH and m=1; or
CH₂CC and m=1; or
CC and m=1; and
R=a group selected from primary amine (NH₂), guanidinium (NHC(═NH)—NH₂), amidine (C(═NH)NH₄),
a secondary amine (NHA, where A=alkyl, e.g. Me, Et, nPr, Bn, preferably lower alkyl, e.g. alkyl 1-4, most preferably Me or Et),
a tertiary amine (NA¹A², where A¹ and A² may be the same or different and are alkyl, e.g. Me, Et, nPr, Bn, preferably Me or Et), or
a quaternary amine (N⁺A¹A²A³ wherein each of A¹, A² and A³ may be the same or different and are alkyl, e.g. Me. Et, nPr, Bn, preferably Me or Et).

24. The conjugate of formula (V) designated αMSH-PPL[99] 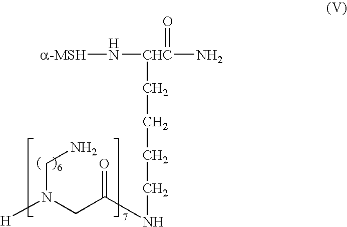
and variants thereof wherein αMSH is substituted by an active derivative thereof or another agent.

25. A conjugate as claimed in claim 24 which is additionally labelled with a label detectable in skin.

26. A conjugate as claimed in claim 25 which carries a fluorescein group attached to the end of the PPL oligomer distant from the conjugated agent.

27. A PNA conjugate of general formula (VI)

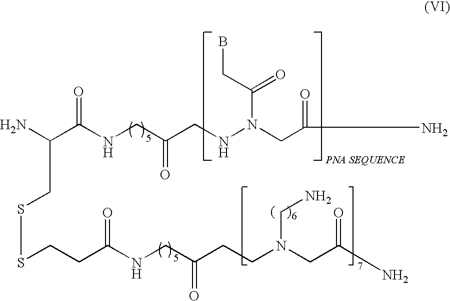

28. A PNA conjugate of claim 27 wherein the PNA targets tyrosinase expression in skin.

29. A PNA conjugate of claim 28 wherein said PNA is the PNA of SEQ, ID no. 1, or an equivalent PNA complementary to human tyrosinase coding sequence.

30. An oligonucleotide conjugate of general formula VII

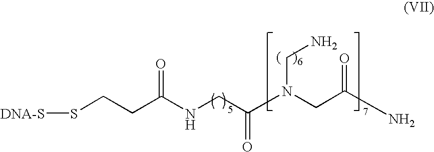

31. An oligonucleotide conjugate of claim 30 wherein said oligonucleotide is an antisense oligonucleotide which targets tyrosinase expression in skin.

32. A method of delivering an agent into skin ex vivo comprising:
(i) providing a conjugate in which the agent is conjugated to poly-peptoid having at least 3 units of formula I 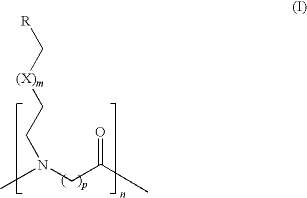
wherein:
p=1 or more, preferably 1, 2 or 4;
X=either CH₂, in which case m=1 or more, e.g. 1 to 5, preferably 3 such that the R group is attached by a hexane side chain; or
CH₂CH═CH and m=1; or
CH═CH and m=1; or
CH₂CC and m=1; or
CC and m=1; and
R=a group selected from primary amine (NH₂), guanidinium (NHC(═NH)—NH₂), amidine (C(═NH)NH₂),
a secondary amine (NHA, where A=alkyl, e.g. Me, Et, nPr, Bn, preferably lower alkyl, e.g. alkyl 1-4, most preferably Me or Et),
a tertiary amine (NA¹A², where A¹ and A² may be the same or different and are alkyl, e.g. Me, Et, nPr, Bn, preferably Me or Et), or
a quaternary amine (N⁺A¹A²A³ wherein each of A¹, A² and A³ may be the same or different and are alkyl, e.g. Me, Et, nPr, Bn, preferably Me or Et)
said conjugate additionally carrying a label detectable in skin;
(ii) applying said conjugate to the cutaneous surface of a skin sample ex vivo, and
(iii) determining whether the labelled conjugate penetrates into the skin sample.

33. The method of claim 32 wherein said agent is conjugated to poly-pseudo-lysine having at least 3 units of formula Ia.

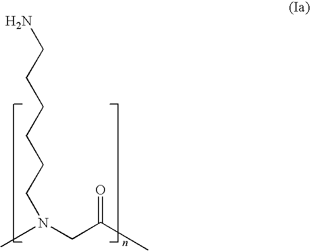

34. The method of claim 32 further comprising determining whether a characteristic of the skin sample is changed by penetration of the conjugate into the sample.

35-36. (canceled)

37. The therapeutic composition of claim 10 wherein said drug is selected form the group consisting of a polypeptide hormone, a peptide, an enzyme, a PNA, polynucleotide or other drug used to treat a skin disorder.

38. The therapeutic composition of claim 10 wherein the poly-peptoid is poly-pseudo-lysine consisting of 7 monomers of formula Ia.

39. The therapeutic composition of claim 10 wherein said conjugate is of general formula II

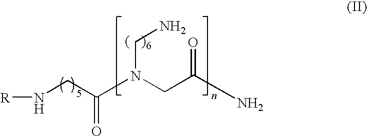

40. The therapeutic composition of claim 39 wherein the drug R is αMSH or an active derivative thereof and n=7 in accordance with formula III

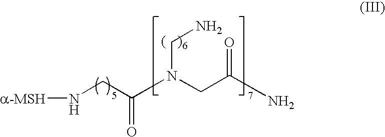

41. The therapeutic composition of claim 10 wherein said conjugate is of general formula IV

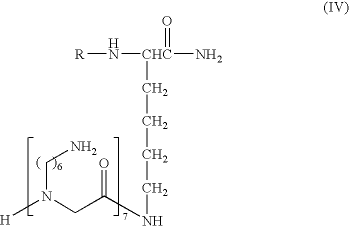

42. The therapeutic composition of claim 41 wherein the drug R is αMSH or an active derivative thereof and n=7 in accordance with formula V

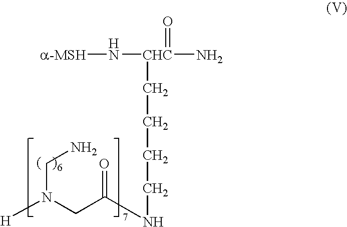

43. The cosmetic preparation of claim 23 in which an agent suitable for changing a skin characteristic is conjugated to poly-pseudo-lysine having at least 3 units of formula Ia

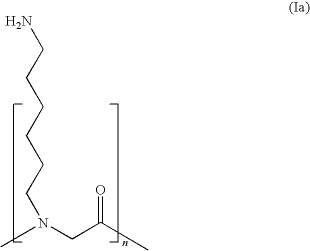

44. The cosmetic preparation of claim 23 wherein the poly-peptoid employed is poly-pseudo-lysine consisting of 7 monomers of formula Ia.

45. The cosmetic preparation of claim 23 wherein said conjugate is of general formula II

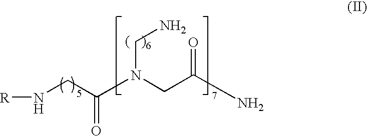

46. The cosmetic preparation of claim 45 wherein the agent is αMSH or a derivative thereof which binds the melancortin 1 receptor (MC1R) and n=7 in accordance with formula III

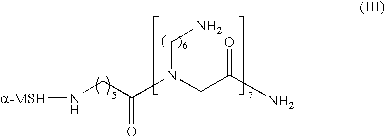

47. The cosmetic preparation of claim 23 wherein said conjugate is of general formula IV

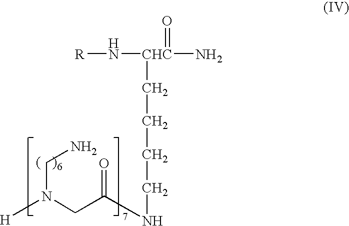

48. The cosmetic preparation of claim 47 wherein the agent is αMSH or a derivative thereof which binds the MC1R and n=7 in accordance with formula V

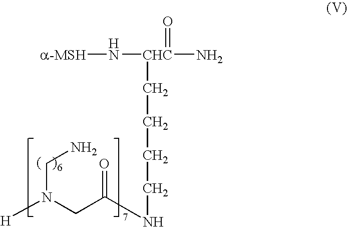

49. The cosmetic preparation of claim 23 wherein said agent is a PNA or antisense olignucleotide which is capable of reducing tyrosinase expression in skin.

50. The cosmetic preparation of claim 49 wherein said PNA is in a conjugate of general formula VI: [chemical structure]


SEQUENCE LISTING (3 sequences)

SEQ ID NO: 1 - DNA, 15 bp, Artificial
  Synthetic construct
gaacattttctcctt

SEQ ID NO: 2 - protein, 13 aa, Artificial Sequence
  Synthetic construct
XYSMEHFRWGKPX

SEQ ID NO: 3 - protein, 13 aa, Artificial Sequence
  Synthetic construct
XYSMEHFRWGKPX